(19) United States
(12) Patent Application Publication
(10) Pub. No.: US 2025/0176101 A1
(43) Pub. Date: May 29, 2025

(54) BONDING STRUCTURE IN ELECTRICALLY CONDUCTIVE PATTERN OF FABRIC

(71) Applicant: WAVE COMPANY CO., LTD., Seoul (KR)

(72) Inventors: Na-Yun CHO, Seoul (KR); Sang-Chul LEE, Seoul (KR); Ji-Hun HAN, Seoul (KR)

(21) Appl. No.: 18/539,666

(22) Filed: Dec. 14, 2023

(30) Foreign Application Priority Data
Nov. 27, 2023 (KR) 10-2023-0166651

(51) Int. Cl. H05K 1/03; H05K 1/11; H05K 3/00

(52) U.S. Cl. CPC H05K 1/038; H05K 1/111; H05K 1/118; H05K 3/0094

(57) ABSTRACT

Disclosed is a bonding technology capable of improving bonding reliability between an electrically conductive pattern on a fabric and a flexible circuit board. A terminal of the flexible circuit board is electrically and physically bonded to a conductive pad by an electrically conductive adhesive interposed therebetween, and the terminal is constituted by a through-hole defined at the flexible printed circuit board and an electrically conductive layer integrated with top and bottom surfaces of the flexible printed circuit board along an edge of the through-hole and with a sidewall of the through-hole. The electrically conductive adhesive is filled into the through-hole and is laminated on the electrically conductive layer in contact therewith.

### REFERENCE TO RELATED APPLICATIONS

This application claims the priority benefit of Korean Patent Application No. 10-2023-0166651 filed on Nov. 27, 2023, the entire contents of which are incorporated herein by reference.

### FIELD OF THE INVENTION

The present invention relates to a bonding structure in an electrically conductive pattern of a fabric, and more particularly, to a technology capable of improving bonding reliability between an electrically conductive pattern on a fabric and a flexible circuit board.

### BACKGROUND OF THE INVENTION

As is well known, electrical stimulation therapy (EST) refers to therapy that artificially induces muscle contraction through electrical signals, and electrical muscle stimulation therapy (EMS), fields of EST, is used as a training tool for athletes and the general public for the recovery of patients who cannot move part of the body or the whole body, as a preventive measure against secondary symptoms, or for athletes' recovery after exercise.

In addition, EMS is beginning to be used for cosmetic purposes in addition to training and treatment. Due to an effect of correcting a body shape as muscles become stronger, EMS is being actively introduced into the field of training for beauty purposes, and as a side effect, high efficiency is shown in calorie consumption.

Moreover, with the popularization of virtual reality 3D content, various digital devices capable of providing more immersive virtual reality services are being developed.

Suits worn by users are also being developed to perform the EMS above or efficiently provide virtual reality services, and a connection structure for reliably transmitting data between a sensor installed in a suit and a wearable device mounted to the suit is a very important variable.

Typically, a line-shaped electrically conductive pattern for electrical connection is on a suit. For example, a silicone rubber coating fabric with an electrically conductive layer formed is disclosed in Korean Patent No. 10-2051137 by the inventor of the present invention.

As in the patent above, an electrically conductive pattern obtained by applying a liquid mixture of electrically conductive particles dispersed in liquid silicone rubber onto a surface of a textile fabric is electrically and physically connected to a flexible circuit board (FPCB) by an electrically conductive silicone adhesive interposed therebetween.

To be specific, the flexible circuit board extends from a wearable device mounted to a user suit and is electrically connected to an electrically conductive pattern on the user suit. The flexible circuit board is made of a polyimide material, and a terminal is disposed at an end by electroless plating of nickel-gold.

A terminal of the flexible circuit board is electrically and physically bonded to an electrically conductive pattern of a fabric by an electrically conductive silicone adhesive interposed therebetween.

However, according to such a structure, not only a terminal of a flexible circuit board but also the flexible circuit board itself is bonded to an electrically conductive pattern of a fabric by a silicone adhesive, but due to the material characteristics of the flexible circuit board, bonding with a silicone adhesive is difficult.

Therefore, even after performing bonding by interposition of a silicone adhesive, there is a disadvantage that the flexible circuit board is easily separated from the silicone adhesive even by weak force or vibration.

As a result, electrical connection between a flexible circuit board and an electrically conductive pattern cannot be maintained reliably, and reliability further declines, especially in shooting games (FPS) that involve intense movement while a user is wearing a suit.

This disadvantage is more likely to occur in the case of wearable devices having multi-electrodes to be electrically connected to a plurality of electrically conductive patterns, and may be further aggravated when the widths of the electrically conductive patterns are reduced and the amount of a silicon adhesive interposed is small.

### SUMMARY OF THE INVENTION

An object of the present invention is to provide a bonding structure in which bonding reliability between an electrically conductive pattern and a flexible circuit board is improved.

Another object of the present invention is to provide a bonding structure in which reliability of electrical and physical connection between a flexible circuit board extending from a device and an electrically conductive pattern on a fabric.

According to an aspect of the present invention, there is provided a bonding structure in which a terminal of a flexible circuit board is bonded to a conductive pad disposed at an end of an electrically conductive pattern on a fabric, wherein the terminal is electrically and physically bonded to the conductive pad by an electrically conductive adhesive interposed therebetween, the terminal is constituted by a through-hole defined at the flexible printed circuit board and an electrically conductive layer integrated with top and bottom surfaces of the flexible printed circuit board and a sidewall of the through-hole along an edge of the through-hole, and the electrically conductive adhesive is filled into the through-hole and is laminated on the electrically conductive layer in contact therewith.

The electrically conductive adhesive may extend over a width of the electrically conductive layer and be bonded to top and bottom surfaces of the flexible circuit board.

An electrically conductive pattern portion including the conductive pad, a flexible circuit board portion including the terminal, and the electrically conductive adhesive may be covered by a protective cover.

The protective cover may be bonded onto the fabric by an electrically insulating adhesive interposed therebetween.

The protective cover may be constituted by a cotton material base sheet and a polyurethane film heat-pressed and bonded to the base sheet, and the base sheet may be bonded to the fabric by the electrically insulating adhesive interposed therebetween.

### BRIEF DESCRIPTION OF THE DRAWINGS

The above objects and other advantages of the present invention will become more apparent by describing in detail exemplary embodiments thereof with reference to the attached drawings in which:

FIG. 1 shows a bonding structure of an electrically conductive pattern of a fabric according to an embodiment of the present invention;

FIGS. 2A and 2B are cross-sectional views taken along line 2-2 in FIG. 1, respectively;

FIG. 3 shows a bonding structure of an electrically conductive pattern of a fabric according to another embodiment of the present invention; and

FIG. 4 is a cross-sectional view taken along line 4-4 in FIG. 3.

### DETAILED DESCRIPTION OF THE INVENTION

It should be noted that the technical terms used in the present invention are only used to describe specific embodiments and are not intended to limit the present invention. In addition, unless specifically defined otherwise in the present invention, the technical terms used in the present invention should be interpreted in the sense generally understood by a person of ordinary skill in the technical field to which the present invention pertains, and should not be interpreted in an overly comprehensive sense or in an excessively reduced sense. Moreover, if a technical term used in the present invention is an incorrect technical term that does not accurately express the idea of the present invention, it should be understood by replacement with a technical term that can be properly understood by those skilled in the art. Furthermore, general terms used in the present invention should be interpreted according to the definition in the dictionary or according to the context, and should not be interpreted in an excessively reduced sense.

Hereinafter, specific embodiments of the present invention will be described in detail with reference to the accompanying drawings.

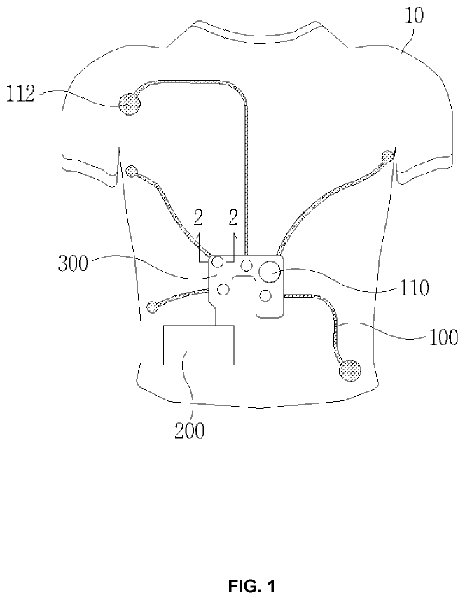

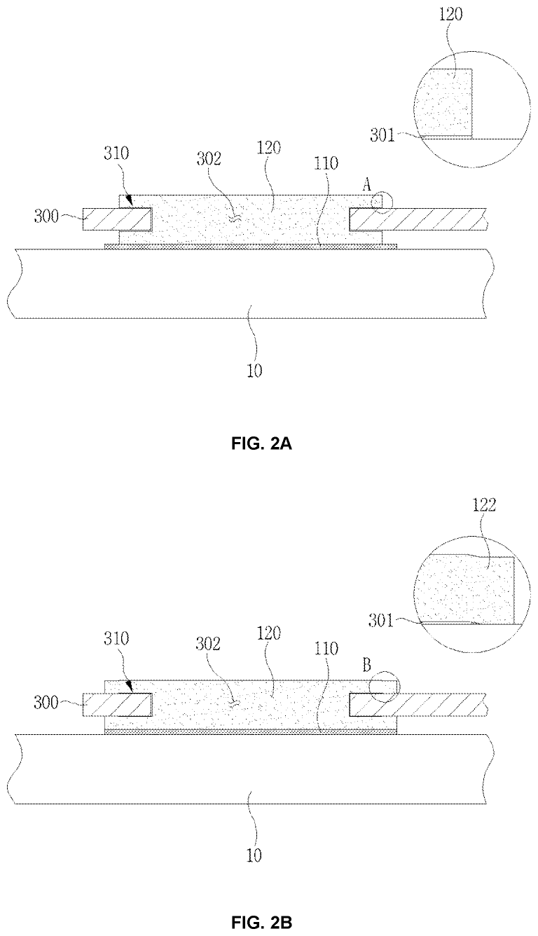

FIG. 1 shows a bonding structure of an electrically conductive pattern of a fabric according to an embodiment of the present invention, and FIGS. 2A and 2B are cross-sectional views taken along line 2-2 in FIG. 1, respectively.

A plurality of electrically conductive patterns 100 are defined on a surface of a suit 10 worn by a user, and conductive pads 110 and 112 are disposed on opposite ends of the electrically conductive patterns 100.

Here, surfaces of the electrically conductive patterns 100 are insulated with silicone coating, and the conductive pads 110 and 112 mean portions located at opposite ends of the electrically conductive patterns 100 and electrically bonded to a flexible circuit board 300 while having surfaces that are not insulated.

The bonding structure of the present invention is particularly needed when the plurality of electrically conductive patterns are required in accordance with the application of wearable devices having multi-electrodes and widths of the electrically conductive patterns are accordingly reduced.

A wearable device 200, such as a controller with a wireless transmission function, is installed in the suit 10, and is electrically connected to the plurality of electrically conductive patterns 100 through one flexible circuit board 300.

To this end, in the a plurality of electrically conductive patterns 100, the conductive pad 110 at one end is gathered at a position adjacent to the device 200, and the conductive pad 110 at the other end is disposed at a position for data input and output.

As is well known, the electrically conductive patterns 100 may be defined by applying a paste mixed with electrically conductive particles or powder, such as silver-coated copper powder, to liquid silicone rubber and hardening the same.

A fabric constituting the suit 10 may include a textile, a knitted fabric, a non-woven fabric, or a knitting fabric, and nylon, polyester, polyurethane, polyimide, or other polymer materials may be applied.

Referring to FIG. 1, one end of the flexible circuit board 300 is electrically connected to the device 200 and extends to intersect the conductive pad 110 of each electrically conductive pattern 100.

Although not shown, in the flexible circuit board 300, the plurality of conductive lines that are in contact with each conductive pad 110 and are electrically connected are disposed to be spaced.

A through-hole 302 is defined at the flexible circuit board 300 in response to the conductive pad 110 disposed at an end of each electrically conductive pattern 100, and an electrically conductive layer 301 is integrated with top and bottom surfaces of the flexible circuit board 300 along an edge of the through-hole 302 and with a sidewall of the through-hole 302 to form a terminal 320.

The electrically conductive layer 301 may be a metal thin film or a metal plating layer, and disposed on the flexible circuit board 300 to be electrically connected to a conductive line extending from the device 200.

Referring to FIG. 2A, the terminal 320 is electrically and physically bonded to the conductive pad 110 by an electrically conductive silicone adhesive 120 interposed therebetween, and the silicone adhesive 120 is filled into the through-hole 302 and is laminated on and bonded to the electrically conductive layer 301.

As a result, the silicone adhesive 120 has a bobbin shape in cross section, so that it is possible to reliably prevent the flexible circuit board 300 from being torn and separated from the silicone adhesive 120 due to a silicone adhesive portion bonded to the electrically conductive layer 301 disposed on top and bottom surfaces of the flexible circuit board 300.

In this embodiment, the silicone adhesive 120 is only bonded to the electrically conductive layer 301 disposed on top and bottom surfaces of the flexible circuit board 300, so that a width of the silicone adhesive 120 is the same as a width of the electrically conductive layer 301. Meanwhile, referring to FIG. 2B, the silicone adhesive 122 extends over a width of the electrically conductive layer 301 and is bonded to top and bottom surfaces of the flexible circuit board 300.

According to such a structure, the cross-sectional shape of an electrically conductive adhesive interposed between a terminal of a flexible circuit board and a conductive pad of an electrically conductive pattern of a fabric is in a bobbin shape, and thus it is possible to prevent the flexible circuit board from being physically torn and separated from the electrically conductive adhesive due to an adhesive bonded to a surface of the flexible circuit board. As a result, it is possible to improve reliability of electrical connection between the flexible circuit board and the electrically conductive pattern.

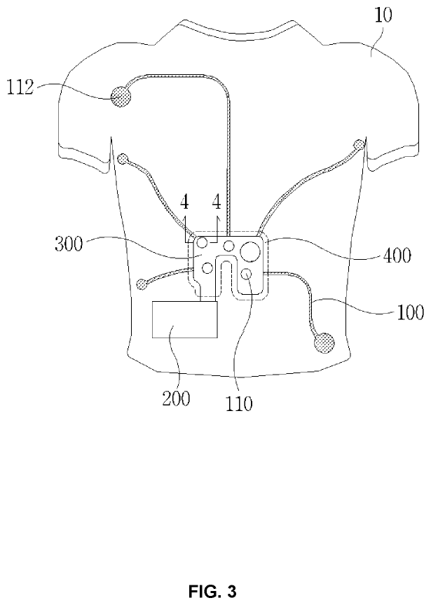

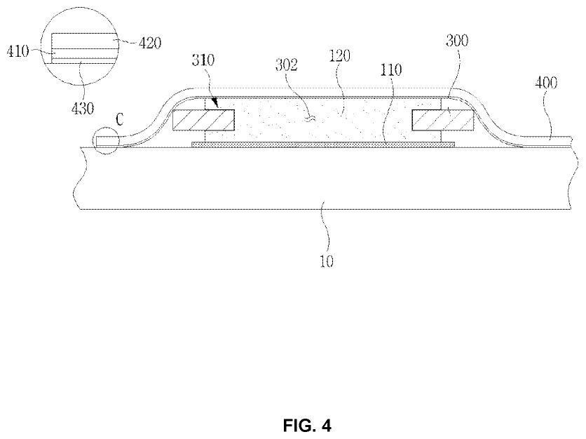

FIG. 3 shows a bonding structure of an electrically conductive pattern of a fabric according to another embodiment of the present invention, and FIG. 4 is a cross-sectional view taken along line 4-4 in FIG. 3.

According to this embodiment, an electrically conductive pattern portion including a conductive pad 110, a flexible circuit board portion including a terminal 310, and a silicone adhesive 120 are covered by a protective cover 400.

As shown at an enlarged circle in FIG. 4, the protective cover 400 is constituted by a cotton material base sheet 410, and a polyurethane film 420 heat-pressed and bonded to the base sheet 410, and the base sheet 410 is bonded to a fabric 10 by an electrically insulating adhesive 430 interposed therebetween.

The polyurethane film 420 is thermally transferred and laminated onto the base sheet 410, making it easier to perform processing, such as cutting, on the base sheet 410.

According to such a structure, by covering an electrically conductive pattern portion, a flexible circuit board portion, and a silicone adhesive by a single protective cover, it is possible to fundamentally prevent a flexible circuit board from being torn and separated from the silicone adhesive.

According to the present invention, bonding reliability between a conductive pad of an electrically conductive pattern on a suit fabric and a terminal of a flexible circuit board can be improved.

In addition, reliability of electrical and physical connection with an electrically conductive pattern of a fabric can be improved by defining a through-hole at a flexible circuit board extending from a device and disposing a terminal at the through-hole so that an electrically conductive adhesive is bonded to the terminal.

A person skilled in the art to which the present invention pertains can make modifications and changes to the description above without departing from the essential characteristics of the present invention. Accordingly, the embodiments disclosed in the present invention are not intended to limit the technical idea of the present invention but are for illustrative purposes, and the scope of the technical idea of the present invention is not limited by these embodiments. The scope of protection of the present invention should be interpreted in accordance with the claims below, and all technical ideas within the equivalent scope should be construed as being included in the scope of rights of the present invention.

What is claimed is:

1. A bonding structure in which a terminal of a flexible circuit board is bonded to a conductive pad disposed at an end of an electrically conductive pattern on a fabric for a suit,
wherein the terminal is electrically and physically bonded to the conductive pad by an electrically conductive adhesive interposed therebetween,
the terminal is constituted by a through-hole defined at the flexible printed circuit board and an electrically conductive layer integrated with top and bottom surfaces of the flexible printed circuit board along an edge of the through-hole and integrated with a sidewall of the through-hole, and
the electrically conductive adhesive is filled into the through-hole and is laminated on the electrically conductive layer in contact therewith.

2. The bonding structure in the electrically conductive pattern on the fabric of claim 1, wherein the electrically conductive adhesive extends over a width of the electrically conductive layer and is bonded to top and bottom surfaces of the flexible circuit board.

3. The bonding structure in the electrically conductive pattern on the fabric of claim 1, wherein an electrically conductive pattern portion including the conductive pad, a flexible circuit board portion including the terminal, and the electrically conductive adhesive are covered by a protective cover.

4. The bonding structure in the electrically conductive pattern on the fabric of claim 3, wherein the protective cover is bonded onto the fabric by an electrically insulating adhesive interposed therebetween.

5. The bonding structure in the electrically conductive pattern on the fabric of claim 3, wherein the protective cover is constituted by a cotton material base sheet and a polyurethane film heat-pressed on and bonded to the base sheet, and
the base sheet is bonded to the fabric by the electrically insulating adhesive interposed therebetween.

6. A smart clothing fabric with a plurality of electrically conductive patterns respectively bonded to a plurality of terminals of a flexible circuit board,
wherein the terminals are electrically and physically bonded to conductive pads disposed at opposite ends of the electrically conductive patterns by an electrically conductive adhesive interposed therebetween,
each of the terminals is constituted by a through-hole defined at the flexible printed circuit board and an electrically conductive layer integrated with top and bottom surfaces of the flexible printed circuit board along an edge of the through-hole and integrated with a sidewall of the through-hole, and
the electrically conductive adhesive is filled into the through-hole and is laminated on the electrically conductive layer in contact therewith.
